(19) United States
(12) Patent Application Publication
(10) Pub. No.: US 2024/0358542 A1
(43) Pub. Date: Oct. 31, 2024

(54) URINE COLLECTION CONTAINER LINER ASSEMBLY AND RELATED SYSTEMS AND METHODS

(71) Applicant: PUREWICK CORPORATION, Covington, GA (US)

(72) Inventors: Tamika Richardson, Schenectady, NY (US); Meghan Caracciola, Washington, DC (US); Padraig Fogarty, Ballina-Killaloe (IR); Anthony Garvey, Listowel (IR); Vinayaka Kulkarni, Bangalore (IN); Shahab Siddiqui, Lawrenceville, GA (US); Ronald Nicolas Legaspi, Alpharetta, GA (US)

(21) Appl. No.: 18/682,006

(22) PCT Filed: Aug. 8, 2022
(86) PCT No.: PCT/US2022/039714

§ 371 Date: Feb. 7, 2024

Related U.S. Application Data
(60) Provisional application No. 63/230,897, filed on Aug. 9, 2021.

(51) Int. Cl. A61F 5/451; A61F 5/44

(52) U.S. Cl. CPC A61F 5/451; A61F 5/4405; A61F 2005/4402

(57) ABSTRACT

Examples relate to urine collection container liner assemblies, and related systems and methods. A urine collection system includes a urine collection device configured to collect urine discharged from a user, a pump configured to draw urine from the urine collection device by applying at least a partial suction force to a reservoir within the urine collection device, a urine collection container in fluid communication with urine collection device, and a liner assembly configured to be disposed within the urine collection container. The liner assembly includes a liner extending from the lid to a closed bottom portion and defining an interior of the liner assembly.

### CROSS-REFERENCE TO RELATED APPLICATIONS

This application claims priority to U.S. Provisional Patent application No. 63/215,017 filed on Aug. 9, 2021, entitled URINE COLLECTION CONTAINER LINER ASSEMBLY, AND RELATED SYSTEMS AND METHODS, the disclosure of which is incorporated by reference herein, in its entirety.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to urine collection container liner assemblies, and related systems and methods. In an embodiment, a liner assembly may be configured to be disposed within a urine collection container. The liner assembly may include a lid configured to be removably attached the urine collection container and a liner configured to extend from the lid into the urine collection container, the liner defining an interior of the liner assembly. The lid may include a vacuum port and a collection port. The vacuum port and the collection port may be configured to be in direct fluid communication with the interior of the liner assembly.

In an embodiment, a urine collection system may be disclosed. The urine collection system may include a urine collection device configured to collect urine discharged from a user, a pump configured to draw urine from the urine collection device by applying at least a partial suction force to a reservoir within the urine collection device, a urine collection container in fluid communication with urine collection device, and a liner assembly configured to be disposed within the urine collection container. The liner assembly may include a liner extending from the lid to a closed bottom portion and defining an interior of the liner assembly.

In an embodiment, a method of collecting urine includes inserting a liner within a urine collection container. The method also includes positioning a urine collection device at least proximate to a urethra of a user. The method also includes receiving urine discharged from the user in the urine collection device. The method also includes receiving urine from the urine collection device in the liner disposed within the urine collection container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a urine collection system, according to an embodiment.

FIG. 1B is a front view of a urine collection system, according to an embodiment.

FIG. 2A is a side isometric view of a liner assembly for a urine collection system, according to an embodiment.

FIG. 2B is a cross-sectional view of a liner assembly for a urine collection system shown with a urine-solidifying film thereon, according to an embodiment.

FIG. 3 is a flow diagram of a method for collecting urine, according to an embodiment.

### DETAILED DESCRIPTION

Embodiments disclosed herein are related to urine collection containers and systems including liner assemblies, and related methods. The devices and systems disclosed herein are configured to collect fluids from an individual. The fluids collected by the fluid collection devices may include at least one of urine, vaginal discharge, penile discharge, reproductive fluids, blood, sweat, or other bodily fluids.

In conventional urine collection systems, the urine is collected in a urine collection container that can then be emptied and washed. Emptying and washing the urine collection container after every use can be an unpleasant, tedious, time consuming, and frustrating chore for the user and/or a caregiver.

To improve conventional urine collection systems, liner assemblies described herein are positioned or positionable in the urine collection container of a urine collection system. In some embodiments, the liner assembly may be inserted into the canister to make removal of the urine much simpler and easier to perform, thereby minimizing the inconvenience of cleaning the system and also minimizing odors common to conventional urine collection systems. The liner assembly may instead prevent the urine from contacting the interior surfaces of the urine collection container and the liner may be disposable in some embodiments.

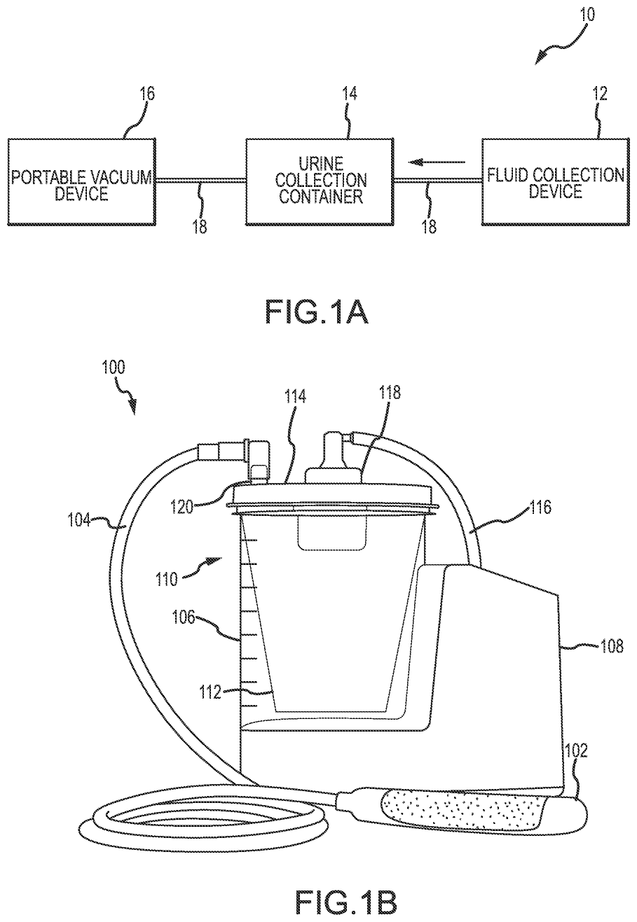

FIG. 1A is a block diagram of a fluid collection system 10, according to an embodiment. The urine collection system 10 may be included in embodiments of fluid collection systems described herein. The system 10 includes a urine collection device 12 (e.g., any of the urine collection devices disclosed herein), a urine collection container 14, and a pump 16 (or vacuum source). The urine collection device 12, the urine collection container 14, and the pump 16 may be fluidly coupled to each other via one or more conduits 18. For example, fluid collection device 12 may be operably coupled to one or more of the urine collection container 14 or the pump 16 via the conduit 18. In some embodiments, the pump 16 may be secured directly to the urine collection container 14. Fluid (e.g., urine or other bodily fluids) collected in the fluid collection device 12 may be removed from the fluid collection device 12 via the conduit 18 secured to the fluid collection device 12. Suction force may be introduced into the chamber of the fluid collection device 12 via the inlet of the conduit 18 responsive to suction (e.g., vacuum) force applied at the outlet of the conduit 18.

The suction force may be applied to the outlet of the conduit 18 by the pump 16 either directly or indirectly. The suction force may be applied indirectly via the urine collection container 14. For example, the outlet of the conduit 18 may be disposed within or fluidly coupled to an interior region of the urine collection container 14 and an additional conduit 18 may extend from the urine collection container 14 to the pump 16. Accordingly, the pump 16 may apply suction to the fluid collection device 12 via the urine collection container 14. The suction force may be applied directly via the pump 16. For example, the outlet of the conduit 18 may be disposed within the pump 16. An additional conduit 18 may extend from the pump 16 to a point outside of the fluid collection device 12, such as to the urine collection container 14. In such examples, the pump 16 may be disposed between the fluid collection device 12 and the urine collection container 14.

The urine collection container 14 is sized and shaped to retain a fluid therein. The urine collection container 14 may include a bag (e.g., drainage bag), a bottle, a canister, or a cup (e.g., collection jar), or any other enclosed container for storing bodily fluid(s) such as urine. In some examples, the conduit 18 may extend from the fluid collection device 12 and attach to the urine collection container 14 at a first point therein. An additional conduit 18 may attach to the urine collection container 14 at a second point thereon and may extend and attach to the pump 16. Accordingly, a vacuum (e.g., suction) may be drawn through fluid collection device 12 via the urine collection container 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

FIG. 1B shows an example of a urine collection system 100, according to an embodiment. The urine collection system 100 may include a urine collection device 102, a first tube 104 in fluid communication with an interior region of the urine collection device 102, a urine collection container 106, and a pump 108. In some embodiments, the urine collection container 106 may include a rectangular shape. The rectangular shape may make the urine collection system 100 more portable. The rectangular shape may make the urine collection container 106 easier to drain and/or clean, in some embodiments. The urine collection system 100 may be made to be disposed in a carrying case or bag, such as a backpack. In some embodiments, the urine collection system 100 may be rectangular shaped.

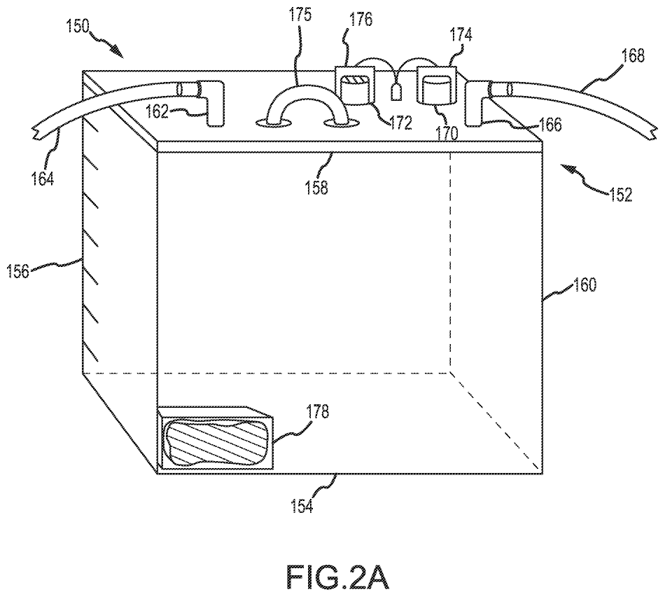

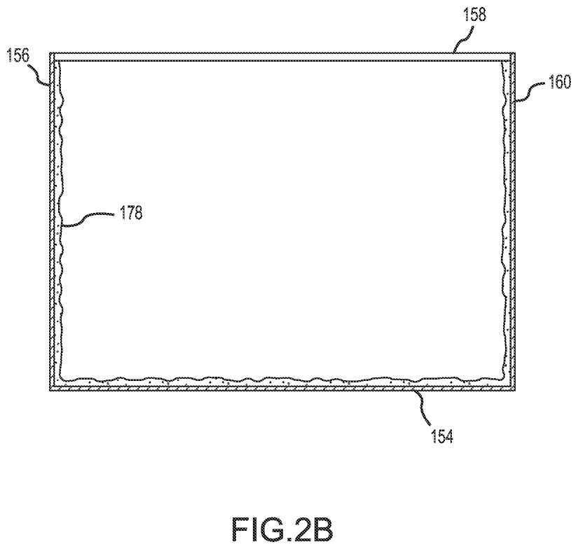

The urine collection container 106 may include a liner assembly 110, in some embodiments. The liner assembly 110 may include a liner 112 and a lid 114 secured or securable to the canister 114. The liner 112 may include a top region and a bottom region, and may at least partially define an interior region in fluid communication with the internal region of the urine collection device 102 via the first tube 104. In some embodiments, the liner 112 may include the same shape as the urine collection container 106. In other words, an outer surface of the liner 112 may completely contact an inner surface of the urine collection container 106. In other embodiments, the liner 112 may include other shapes, where the liner 112 does not contact the inner surface of the urine collection container 106 completely. In some embodiments, the liner 112 may rest on a rim of the urine collection container 106 and not contact the inner surface of the urine collection container 106. In some embodiments, the liner 112 may be rectangular or cylindrical shaped. In some embodiments, as shown in FIG. 2A, the liner 112 may include a wider upper portion that narrows to a closed bottom portion. For example, the liner 112 can extend from the lid 114 to the closed bottom portion, the liner defining an interior of the liner assembly 110. In some embodiments, the lid 114 may be configured to be removably attached to an upper portion of the urine collection container 106.

The use of liner 112 can reduce cleaning frequency of the urine collection container 106, odors, and also makes a reduced workload on the user of the urine collection system 100 and/or a caretaker. Cleanliness can be improved without affecting the design and/or operation of the permanent components of the urine collection system 100. In some embodiments, the liner 112 may be configured to be reusable. The urine can be drained into a waste receptacle and the liner may be cleaned and returned to the urine collection container 106. In other embodiments, the liner 112 may be configured to be removable and replaceable. For example, the liner 112 may be single-use. After the urine collection container 106 has been filled once, the liner 112 may be disposed of and a new liner 112 may be placed within the urine collection container 106. The liner 112 can be disposed of along with the urine, which reduces urine exposer to the user and/or caretaker.

In some embodiments, the liner 112 may be configured to be non-collapsible under a vacuum pressure suitable to draw urine from the urine collection device 102 through the first tube 104 and into the urine collection container 106. Thus, the liner may be configured to be non-collapsible under a vacuum pressure of up to about 300 mmHg. For example, the liner 112 may be non-collapsible under a vacuum pressure of about 30 mmHg to about 60 mmHg, about 60 mmHg to about 100 mmHg, about 100 mmHg to about 150 mmHg, about 150 mmHg to about 200 mmHg, about 200 mmHg to about 300 mmHg, greater than about 50 mmHg, greater than about 100 mmHg, greater than about 200 mmHg, greater than about 250 mmHg, less than about 300 mmHg, less than about 250 mmHg, less than about 200 mmHg, less than about 150 mmHg, less than about 100 mmHg, or less than about 50 mmHg.

In some embodiments, the liner 112 can include a durable material that can be economical to dispose of and/or recycle. In some embodiments, the liner 112 can include at least one of a durable polyethylene, a cardstock, or a cardboard. In some embodiments, the liner 112 can include a soft polyethylene material and/or composite. In other embodiments, the liner 112 can include a cardstock having a weight from about an equivalent of light weight paper to about an equivalent of extra-heavy weight paper. In some embodiments, the cardstock may include a weight from about 65 lb. cardstock to about 140 lb. cardstock. In some embodiments, the liner 112 can include cardboard having a single face board, a sing wall board, a double wall board or a combination thereof.

The urine collection device 102 may be configured to be positioned at least proximate to a urethra of a user. While the urine collection device 102 shown in FIG. 1B includes a female urine collection device, the urine collection device 102 may instead include a male urine collection device. PCT International Application No. PCT/US2019/029616, for example, describes various embodiments of both male and female fluid collection devices, the disclosure of which are incorporated in their entirety by this reference. Moreover, the urine collection device 102 may be interchangeable in the urine collection system 100 between different types, varieties, and sizes of male or female urine collection devices. Generally, the urine collection device 102 may include a surface sized to be positioned proximate or adjacent to the urethra and configured to wick urine or other fluids away from the user. Urine or other fluids may be wicked from the surface to a reservoir in the urine collection device 102.

The urine collection system 100 also includes the first tube 104 in fluid communication with an interior region (e.g. reservoir) of the urine collection device 102 and the interior region of the urine collection container 106. The first tube 104 may be positioned between the urine collection device 102 and the urine collection container 106. The urine collection system 100 also may include a second tube 116 providing fluid communication between the pump 108 and the interior region of the urine collection container 106. In some embodiments, the pump 108 may be secured directly to the urine collection container 106, and the second tube 116 may be absent from the urine collection system 100. The tubes 104, 116 may include a flexible tube.

The urine collection container 106 may be reusable and dishwasher safe, and may include a generally rigid material such as polycarbonate or glass. In some embodiments, the urine collection container 106 may be disposable. The urine collection system 100 may be configured to accommodate different volumetric sizes of urine collection containers 106. In some embodiments, the urine collection container 106 may include at least one of a volume having 500 mL, 800 mL, 1000 mL, or 2000 mL. In some embodiments, the lid 114 may include multiple ports including a vacuum port 118 and a collection port 120. In some embodiments, the lid 114 may include additional ports or adapters than those shown in FIG. 1B. The lid 114 may include a dispensing spout and a vent spout in fluid communication with the interior of the liner assembly 110. Except for the ports and/or adapters for attachment of the tubes 104, 116, the urine collection container 106 may be sealed and airtight such that the urine collected in the urine collection container 106 does not leak or spill.

The pump 108 is in fluid communication with the interior region of the urine collection container 106 and is configured to pull a vacuum on the interior region of the urine collection container 106 effective to draw the urine from the urine collection device 102 through the first tube 104 into the urine collection container 106. The pump may be configured to draw urine from the urine collection device 102 by applying at least a partial suction force to a reservoir within the urine collection device 102. The pump 108 may be secured directly to the urine collection container 106, or the second tube 116 may fluidly couple the pump 108 with the interior region of the urine collection container 106. The pump 108 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 108 may provide a vacuum or suction to remove fluid from the fluid collection device 108. In some embodiments, the pump may be configured to provide a vacuum pressure of up to about 300 mmHg. In some examples, the pump 108 may be powered by one or more batteries or other power sources. The pump 108 may be included in a housing that also supports the urine collection container 106. In some embodiments, the housing may be rectangular shaped. In some examples, the pump 108 and/or housing may be sized and shaped to fit within a container support on a wheelchair. In some embodiments, the pump 108 may include a wall-mounted pump. The pump 108 may include one or more of a switch, a button, a plug, a remote, or any other device suitable to activate the pump 108.

FIG. 2A is a side isometric view of a liner assembly 150, according to an embodiment. In some embodiments, the liner assembly 150 may be included in urine collection system 100, according to an embodiment. In some embodiments, the liner assembly 150 is removably positionable in the interior region of the urine collection container 106 (not shown in FIG. 2A). When positioned in the interior region of the urine collection container 106, a top portion 152 of the liner assembly 150 is positioned proximate to a top region of the urine collection container 106 and a closed bottom portion 154 of the liner assembly 150 is positioned proximate to a bottom region of the urine collection container 106. An outer surface 156 of the liner assembly 150 may be generally complementary to the interior surface of the one or more sidewalls of the urine collection container 106 when the liner assembly 150 is positioned in the interior region of the urine collection container 106.

In some embodiments, the liner assembly 150 includes a lid 158 configured to be removeably attached to a urine collection container (e.g. urine collection container 106) and a liner 160 configured to extend from the lid 158 to the closed bottom portion 154 and defining an interior of the liner assembly 150. In some embodiments, the lid 158 may include a collection port 162 to which a first tube 164 may be detachably secured to provide fluid communication between a urine collection device and the interior region of the liner assembly 150. A second tube 168 may provide fluid communication between the interior region of the liner assembly 150 and a pump. For example, the lid 158 may include a vacuum port 166 to which a second tube 168 may be detachably connected to provide fluid communication between a pump and the interior region of liner assembly 150. The vacuum port 166 and/or the collection port 162 may include an adapter detachably secured thereto to connect the respective tube 164, 166 to the vacuum port 166 and/or the collection port 162. In some embodiments, at least one (e.g., both) of the first tube 164 and the second tube 168 may be detachably connected or coupled to the lid 158 effective to provide fluid communication with the interior region of the liner assembly 150. For example, the lid 158 may include at least one (e.g. both) of the collection port 162 and the vacuum port 166. In some embodiments, the lid 158 includes a dispensing spout 170 and a vent spout 172. In some embodiments, the lid 158 further includes a first cap 174 configured to cover the dispensing spout 170 and a second cap 176 configured to cover the vent spout 172 when not in use. The first cap 174 and second cap 176 may be connected to the lid 158 with a lanyard and configured such that the caps 174, 176 are removed and the vent spout 172 is open when the fluid is dispensed through the dispensing spout 170, to aid in proper air flow as the fluid is removed. Thus, the liner assembly 150 may be reusable by draining the urine from the dispensing spout 170. The caps 174, 176 maintain the liner assembly 150 air tight to allow the vacuum to pull the urine from the first tube 164 and into the liner assembly.

In some embodiments, at least one of the lid 158 or the liner 160 may be configured to be connected to the urine collection container. In some embodiments, an exterior surface of the liner 160 may include an adhesive. In some embodiments, the lid 158 may connect to the urine collection container with a lip that extends over the urine collection container. In some embodiments, the lid 158 may include clips, straps, fasteners, and/or extensions that connect to an exterior and/or interior surface of the urine collection container. In some embodiments, the liner assembly 150 includes a handle 175 (e.g. a grip) that the user or caregiver may grasp to remove the liner assembly 150 from the urine collection container. The handle 175 may make the liner assembly 150 easier to remove to minimize fluid spills. In some embodiments, the handle 175 may be foldable such that the handle may fit between the urine collection container and the liner assembly 150 or may be low-profile such that the handle 175 does not extend off the lid 158 when not in use.

In some embodiments, the liner assembly 150 further includes a urine-solidifying agent 176. In some embodiments, the urine-solidifying agent 178 includes a packet that can be added to the liner 160. In other embodiments, as shown in FIG. 2B, the urine-solidifying agent 178 includes a film coated on an interior surface of the liner 160. In some embodiments, the urine-solidifying agent 178 may include a high water-absorbent polymer that gelifies the urine. In some embodiments the urine-solidifying agent 178 may be in the form of a powder, granules, flakes, fibers or a film and may be spread throughout the entire interior surface of the liner 160. In some embodiments, the urine-solidifying agent 178 includes N-alkyl- or N-alkylene-substituted acryl and methacryl amides. In other embodiments, the urine-solidifying agent 176 includes sodium plyacrylate. The urine-solidifying agent can reduce odors and improve cleanliness, making the urine easier to remove and dispose of.

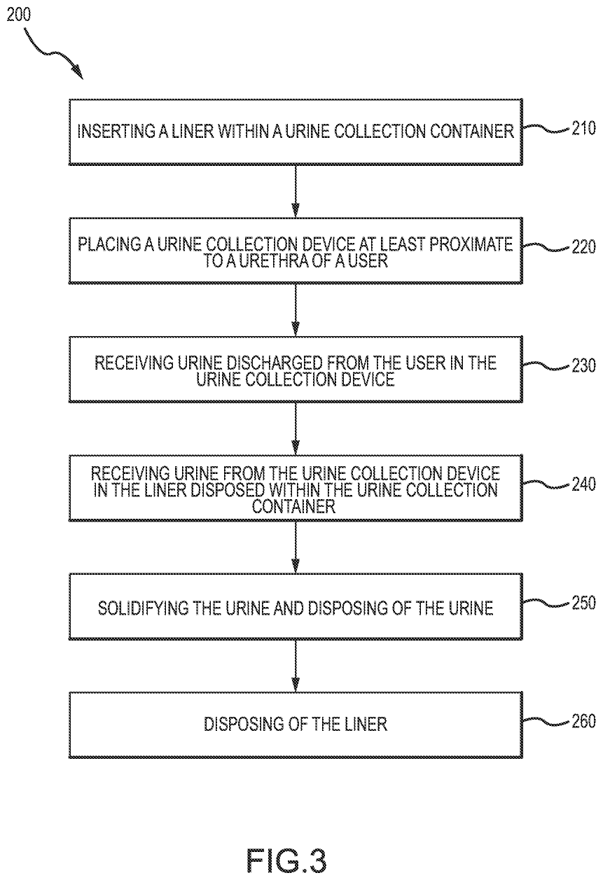

FIG. 3 is a flow diagram of a method 200 for collecting urine, according to an embodiment. The method 200 includes an act 210 of inserting a liner within a urine collection container. In some embodiments, the method 200 includes an act 220 of placing a urine collection device at least proximate to a urethra of a user. In some embodiments, the urine collection device may be in fluid communication with the urine collection container through a first tube. In some embodiments, the method 200 includes an act 230 of receiving urine discharged by the user in the urine collection device. In some embodiments, the method 200 includes an act 240 of receiving urine from the urine collection device in the liner disposed within the urine collection container.

The liner in the method 200 may include any liner and/or liner assembly described herein. In some embodiments, the method 200 may further include an act 250 of solidifying the urine and disposing of the urine. Solidifying the urine may include adding a urine-solidifying agent to the liner as an independent packet. In some embodiments, the urine-solidifying agent includes a film coated on an interior surface of the liner. In some embodiments, the method 200 may further include an act 260 of disposing of the liner. The act 260 may include removing the liner that includes liquid and/or solidified urine and disposing of the liner in a waste receptacle. A new liner can then be inserted into the urine collection container. In some embodiments, the liner can be emptied, cleaned, and reused.

The acts of the method 200 described above are for illustrative purposes. For example, the acts of the method 200 can be performed in different orders, split into multiple acts, modified, supplemented, or combined. In an embodiment, one or more of the act of the method 200 can be omitted. Any of the acts of the method 200 can include using any of the urine collection systems and/or liner assemblies disclosed herein.

As used herein, the term “about” or “substantially” refers to an allowable variance of the term modified by “about” or “substantially” by +10% or +5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A liner assembly configured to be disposed within a urine collection container, the liner assembly comprising:
a lid configured to be removably attached to the urine collection container, the lid including a vacuum port and a collection port; and
a liner configured to extend from the lid into the urine collection container, the liner defining an interior of the liner assembly, wherein the lid further includes a dispensing spout and a vent spout in fluid communication with the interior of the liner and wherein the vacuum port and the collection port are configured to be in direct fluid communication with the interior of the liner assembly.

2. The liner assembly of claim 1, wherein the liner is configured to be non-collapsible under a vacuum pressure of up to about 300 mmHg.

3. The liner assembly of claim 1, wherein the liner includes at least one of a polyethylene, cardstock, or cardboard.

4. The liner assembly of claim 1, wherein at least one of the liner or the lid is configured to be connected to the urine collection container.

5. (canceled)

6. The liner assembly of claim 1, wherein the lid further includes a first cap configured to cover the dispensing spout and a second cap configured to cover the vent spout.

7. The liner assembly of claim 1, further comprising a urine-solidifying agent.

8. The liner assembly of claim 7, wherein the urine-solidifying agent includes a packet included in the interior of the liner.

9. The liner assembly of claim 7, wherein the urine-solidifying agent includes a film coated on an interior surface of the liner.

10. The liner assembly of claim 1, wherein the liner is configured to be reusable.

11. The liner assembly of claim 1, wherein the liner is configured to be disposed after a single use.

12. A urine collection system, comprising:
a urine collection device configured to collect urine discharged from a user;
a pump configured to draw urine from the urine collection device by applying at least a partial suction force to a reservoir within the urine collection device;
a urine collection container in fluid communication with urine collection device; and
a liner assembly configured to be disposed within the urine collection container, wherein the liner assembly includes a liner extending from the lid to a closed bottom portion and defining an interior of the liner assembly; and
a lid configured to be removably attached to an upper portion of the urine collection container, wherein the lid includes a dispensing spout and a vent spout in fluid communication with the interior of the liner assembly.

13. The urine collection system of claim 12, wherein the liner is configured to be removable and replaceable.

14. The urine collection system of claim 12, wherein the liner assembly includes a urine-solidifying agent.

15. The urine collection system of claim 12, wherein the urine collection container includes a rectangular shape.

16. (canceled)

17. The urine collection system of claim 12, wherein the lid further includes a vacuum port configured to be in fluid communication with the pump and a collection port configured to be in fluid communication with the fluid collection device.

18. (canceled)

19. A method of collecting urine, the method comprising:
inserting a liner within a urine collection container;
placing a urine collection device at least proximate to a urethra of a user;
receiving urine discharged from the user in the urine collection device; and
receiving urine from the urine collection device in the liner disposed within the urine collection container; and
emptying the urine from the liner, wherein the urine collection container comprises a lid configured to be removably attached to an upper portion of the urine collection container, wherein the lid includes a dispensing spout and a vent spout in fluid communication with the interior of the liner.

20. The method of claim 19, further comprising solidifying the urine and disposing of the urine.

21. The method of claim 19, further comprising disposing of the liner.
